(19) United States
(12) Patent Application Publication
(10) Pub. No.: US 2007/0276342 A1
(43) Pub. Date: Nov. 29, 2007

(54) Devices and related methods for treating incontinence

(76) Inventors: Bryant Lin, Palo Alto, CA (US); Paul Wang, Saratoga, CA (US); Joseph A. Knight, Palm Harbor, FL (US); Stewart McCallum, Villanova, PA (US); Bertha Chen, Menlo Park, PA (US); Tatum Tarin, Redwood, CA (US)

Correspondence Address: SHAYGLENN LLP, 2755 CAMPUS DRIVE, SUITE 210, SAN MATEO, CA, 94403, US

(21) Appl. No.: 11/729,118

(22) Filed: Mar. 28, 2007

Related U.S. Application Data
(60) Provisional application No. 60/786,279, filed on Mar. 28, 2006.

(51) Int. Cl. A61M 27/00

(52) U.S. Cl. 604/264

(57) ABSTRACT

A device completely disposable within a bodily lumen of a patient and for controlling flow through the bodily lumen having a device body having a proximal end, a distal end and a device lumen within the device body extending from the proximal end to the distal end and a fixation element extending from the outer wall of device body adapted and configured to engage with the wall of the bodily lumen. The device body is adapted and configured to move from a position where the device lumen blocks flow through the bodily lumen to a position where the device lumen allows flow through the bodily lumen. In other configurations, there are one or more valves within the device lumen adapted and configured to move between a position to block flow through the bodily lumen and a position to allow flow through the bodily lumen. There is also a method for implanting a device within a body lumen by placing a device having a device body and fixation elements extending from the device body on a catheter and then dilating the body lumen such that the device body and the fixation elements may advance through the dilated body lumen without the fixation elements engaging the wall of the body lumen. Next, advancing the device to an implant site within the body lumen and then engaging the walls of the body lumen with the fixation elements.

### CROSS REFERENCE TO RELATED APPLICATIONS

This application claims the benefit under 35 U.S.C. §119 of U.S. Provisional Patent Application No. 60/786,279 titled “Wirelessly Controlled Intra-Urethral Urinary Incontinence Device and Delivery and Removal Method” filed Mar. 28, 2006 and U.S. Provisional Patent Application titled Physiologic, Time Delayed Flow Control Device With Delivery and Removal Method” filed May 1, 2006, each of which are incorporated herein by reference in its entirety.

### FIELD OF THE INVENTION

The invention relates to devices for treating incontinence and the methods for their delivery and removal.

### BACKGROUND OF THE INVENTION

Urinary incontinence (UI), the involuntary leaking of urine, presents a large personal and societal burden. Over 17 million people in the United States alone suffer some degree of UI costing the health care system over $20 billion per year. Over 10 million patients in the U.S. suffer from Stress Urinary Incontinence with at least 1 million having some form of intrinsic sphincter deficiency. It is also estimated that at least one half of patients with UI do not report the problem. Both sexes are affected although UI is more common in women especially at a younger age. The prevalence of UI lies anywhere from 20 to 30 percent in young women to over 50 percent in the elderly. Seventeen percent of all males in the U.S. who are over 60 have some degree of incontinence. UI clearly impacts quality of life psychologically and socially interferes with many routine activities. The morbidity related to UI includes cellulitis and ulcers from skin irritation, sleep deprivation, and urosepsis.

There are three primary types of incontinence: Urge Incontinence, Stress Incontinence and Mixed Incontinence. Urge Incontinence occurs with involuntary loss of urine with preceding or simultaneous sensation of urgency. Urge incontinence is attributed to overactive bladder and detrusor (muscle causing bladder contraction) instability. Up to one third of UI is associated with Urge Incontinence. Stress Urinary Incontinence (SUI) is the involuntary leakage on sneezing, coughing or other effort or exertion. It is the most common cause of UI in young women. From 29 to 75% of all urinary incontinence cases in women are due to SUI. Injury during childbirth is a significant risk factor in the development of SUI. Previous pelvic surgery, especially hysterectomy, is associated with SUI. Intrinsic Sphincter Deficiency (ISD), a subset of SUI, is due to a poorly functioning urethral mucosa and/or muscle. The urethral sphincter, located at the neck of the urethra near the bladder, normally constricts and provides coaptation of the urethra and a water-tight seal. With ISD, the urethral closure fails typically due to operative trauma, scarring, and atrophy. Mixed urinary incontinence is associated with BOTH urgency and exertion. This type of UI is now considered the most common type of incontinence covering an overlap of Urge and Stress Incontinence.

Current Treatment for Stress Urinary Incontinence can be classified into three broad areas of non-invasive, surgical, and device based treatment. The non-invasive methods include: Pelvic muscle exercises, behavioral therapy, and medications. Surgical treatments include slings, suspensions, radiofrequency treatment, and urethral bulking (Collagen injection). The implantable or insertable/implantable devices include: External Compression/Occlusive, Catheters, Urethral Prostheses and Inserts (FemSoft (Rochester Medical), ConSert (Conticare) ACT (Uromedica)) and Artificial Urinary Sphincter (American Medical Systems AMS 800). The non-invasive methods are the first line of treatment but have low success rates. The surgical methods have higher “cure” rates (up to 70 or 80 percent) but not all patients are candidates for surgery (especially those older than 70) and some patients refuse surgery. It is believed that current surgical methods do not optimally treat ISD. Device based treatments either replace or bypass the sphincter and so can treat ISD but current devices have many drawbacks. The catheters are uncomfortable and prone to infection. Inserts and prostheses are uncomfortable, prone to infection and require frequent (daily or greater) changes by the patient. Some available devices have a high failure and complication rates are very invasive to place and cause patient discomfort because the continence is maintained by using an inflatable cuff to compress the urethra.

The prior art devices do not address the significant clinical need to provide less invasive devices and methods to prevent incontinence. What is needed are continence devices and methods improve patient comfort, are easily delivered and removed without special skill.

### SUMMARY OF THE INVENTION

In one embodiment of the invention, there is provided a device completely disposable within a bodily lumen of a patient and for controlling flow through the bodily lumen, having a device body having a proximal end, a distal end and a device lumen within the device body extending from the proximal end to the distal end, the device body completely disposed within the bodily lumen so that all flow through the bodily lumen flows through the device lumen; and a fixation element extending from the outer wall of device body adapted and configured to engage with the wall of the bodily lumen; wherein, the device body is adapted and configured to move from a position where the device lumen blocks flow through the bodily lumen to a position where the device lumen allows flow through the bodily lumen. In one aspect, the device body moves in response to increased intraluminal pressure applied anatomically proximal to the device. In another aspect, the increased pressure is applied for longer than three seconds. In another aspect, the increased pressure is applied above a threshold pressure for three to five seconds. In another aspect, at least one of the proximal device end or the distal device end is angled to facilitate movement of the device between positions. In one aspect the angle is less than 90 degrees or placed relative to the device body such that the device body does not form a right cylinder. In another aspect, the fixation element withdraws into the sidewall of the device body or the device lumen. In yet another aspect, the fixation element is a balloon. In another aspect, the fixation element has a stowed condition where the fixation element is engaged with the bodily lumen wall and a deployed condition where the fixation element engages with the bodily lumen wall. In another aspect, the bodily lumen is the urethra. In yet another aspect, the device lumen is non-linear. In another aspect, the device lumen includes baffles.

In another embodiment of the present invention, there is provided a device completely disposable within a bodily lumen of a patient and for controlling flow through the bodily lumen having a device body having a proximal end, a distal end and a device lumen within the device body extending from the proximal end to the distal end, the device body completely disposed within the bodily lumen so that all flow through the bodily lumen flows through the device lumen; and one or more valves within the device lumen adapted and configured to move between a position to block flow through the bodily lumen and a position to allow flow through the bodily lumen. In one aspect, there is also provided a fixation element extending from the outer wall of device body adapted and configured to engage with the wall of the bodily lumen. In another aspect, the one or more valves within the device lumen includes a magnetically activated rotary valve. In another aspect, the one or more valves within the device lumen includes a flap valve. In another aspect, the one or more valves within the device lumen are angled relative to the device lumen. In another aspect, the one or more valves within the device lumen move between a position to block flow through the bodily lumen and a position to allow flow through the bodily lumen by moving a fluid from the interior of a valve to a cavity in the sidewall of the device body. In another aspect, the one or more valves within the device lumen is a valve having a pair of disks mounted on, in or within the sidewall of the bodily lumen, the pair of disks rotatable relative to one another to move between positions.

In another alternative embodiment, there is provided a method for implanting a device within a body lumen including placing a device having a device body and fixation elements extending from the device body on a catheter; dilating the body lumen such that the device body and the fixation elements may advance through the dilated body lumen without the fixation elements engaging the wall of the body lumen; advancing the device to an implant site within the body lumen; and engaging the walls of the body lumen with the fixation elements. In one aspect, the dilating step is performed by inflating a balloon or expanding a structure on the catheter. In another aspect, the balloon is distal to the distal end of the device. In another aspect, the dilating step is performed by inflating a balloon or expanding a structure on the device. In another aspect, the engaging step is performed by rotating the device relative to the bodily lumen. In yet another aspect, each of the valves opens at a different pressure set point or under a different pressure response profile depending upon location within the device lumen. In yet another aspect, the response of each valve of the one or more valves may be adjusted while the device is implanted within the body lumen. In another aspect, the response of the valves is adjusted by altering the magnetic field used to control the response of the valves. In one aspect, the magnetic field is altered by inserting a spacing device between magnets that form a magnetic pair.

In additional alternative aspects and embodiments of the invention, there is provided an incontinence device completely disposable within a bodily lumen of a patient and for controlling flow through the bodily lumen having an elongate member having a proximal end, a distal end and a lumen within the elongate member extending from the proximal end to the distal end, the elongate member completely disposed within the bodily lumen so that all flow through the bodily lumen flows through the elongate member lumen; a fixation element extending from the outer wall of elongate body adapted and configured to engage with the walls of the bodily lumen; and a liner extending along and attached to the elongate member lumen moveable between a normally closed position to prevent flow though the elongate body lumen and an open position to allow flow through the elongate member lumen.

In one aspect, the liner is moveable using physiologic pressure. In another aspect, the liner is moved using an intraluminal pressure applied anatomically distal to the elongate body. In another aspect, the elongate member is moveable between a normally closed position to prevent flow though the elongate member lumen and an open position that allows flow through the elongate member lumen. In another aspect, the elongate member is canted with angled proximal and distal ends. In another aspect, the liner contains a viscous fluid. In another aspect, the elongate member lumen is held in a normally closed position using magnets within the liner wherein the physiologic force overcome the magnetic force to allow flow through the elongate member lumen. In one aspect, the magnets compress the liner into a closed position. In another aspect, the magnets constrict the liner into a closed position using a cuff that extends at least partially around the liner. In yet another aspect, the liner is maintained in a normally closed position by a material contained within the liner. In one aspect, the material is a magnetorheologic fluid. In another aspect, the material is a hydrogel. In another aspect, the material is an electrorheologic material. In another aspect, the material in the liner is a material that changes viscosity when subjected to an external stimulus. In one aspect, the stimulus is intraluminal or intraurethral pressure applied anatomically distal to the device. In another embodiment, the external stimulus is a temperature stimulus, an electrical field stimulus, a magnetic field stimulus, a chemical stimulus or an ultrasonic stimulus. In another aspect, the external stimulus is provided by a device external to the body without a physical connection to any component of the device. In another aspect, the liner comprises two or more liner portions attached to different segments of the elongate member lumen. In one aspect, the bodily lumen is a portion of a vein, a portion of the urethra, a portion of the colon, a portion of the esophagus or a portion of the alimentary canal.

In yet another aspect, the fixation technique used to secure the elongate body within the bodily lumen further includes a fixation element at the proximal end and the distal end of the device, the fixation elements disposed within the bodily lumen except where the fixation elements penetrate the wall of the bodily lumen. In another aspect, the fixation element has a stowed condition where it does not engage with the bodily lumen wall and a deployed condition where the fixation element does engage with the bodily lumen wall. In one aspect, when the fixation element is in the stowed condition the fixation element is in, on or within the elongate body side wall or within the elongate body lumen. In another aspect, the fixation element moves from a condition unengaged with the bodily lumen wall to a condition engaged with the bodily lumen wall by rotating the elongate member. In one aspect, the elongate body rotates less than a full turn, less than half a turn, or less than one-quarter turn.

### BRIEF DESCRIPTION OF THE DRAWINGS

FIG. 1 is a chart illustrating the relationship between proximal intraurethral pressure and flow;

FIG. 2 is a section view of a body lumen with a flow device embodiment;

FIG. 3 is a section view of a body lumen with expanding the flow device of FIG. 3 and deploying fixation elements into the body lumen;

FIGS. 3A and 3B illustrate a section view of a device with retractable fixation devices in a stowed and deployed condition, respectively;

FIGS. 3C and 3D illustrate a section view of a device with another retractable fixation element embodiment in a stowed and deployed condition, respectively;

FIG. 4 illustrates the a catheter being withdrawn from the device of FIG. 3;

FIG. 5 illustrates flow pressure increasing within the lumen 105 while the device remains closed;

FIGS. 6 and 7 illustrate the device in FIG. 5 with increasing lumen pressure;

FIG. 8 illustrates the device of FIG. 5 in a fully open condition;

FIG. 9 illustrates the device of FIG. 5 in a fully closed position inside the body lumen;

FIGS. 10 and 11 are side and end views of the device of FIG. 8;

FIGS. 12 and 13 are side and end views of the device of FIG. 9;

FIGS. 14A and 14B are sagittal and coronal plane views, respectively, of the female abdomen;

FIGS. 14C and 14D are sagittal and coronal plane views, respectively, of the male abdomen;

FIGS. 15, 16 and 17 are section views of a magnetically closed liner device in the closed, partially open and open conditions, respectively;

FIGS. 18 and 19 illustrate partial section views of a magnetic cuff closing device in open and closed conditions, respectively;

FIGS. 20 and 21 illustrate partial section views of another magnetic cuff closing device in open and closed conditions, respectively;

FIGS. 22A, 22B, 22C, 22D and 23 illustrate various techniques for adjusting magnetic closing forces;

FIGS. 24 and 25 illustrate section views of a device having a liner that opens (FIG. 25) and closes (FIG. 24) by moving fluid into and from a diaphragm in the device sidewall;

FIGS. 26 and 27 illustrate section views of a device having an annulus that opens (FIG. 27) and closes (FIG. 26) by moving fluid into and from a piston the device sidewall;

FIGS. 28 and 29 illustrates a flow control device in a body lumen in a closed condition (FIG. 28) and an open condition (FIG. 29);

FIGS. 30 and 31 illustrates a flow control device in a body lumen in a closed condition (FIG. 30) and an open condition (FIG. 31);

FIG. 32 illustrates a section view of a flow control device with two control valves;

FIG. 32A illustrates a flow control valve of FIG. 32 modified to include a magnetic closure;

FIGS. 33A and 33B illustrate section and end views, respectively, of a rotary control valve in a closed condition;

FIGS. 34A and 34B illustrate section and end views, respectively, of a rotary control valve in an open condition;

FIG. 35 illustrates a patient using an external control device to operate an implanted flow control device;

FIGS. 36 and 37 are section views of a flow control device using ligaments to control flow in a closed condition (FIG. 36) and an open condition (FIG. 37);

FIGS. 38 and 39 are section views of a flow control device using rolled control elements shown in a closed condition (FIG. 38) and an open condition (FIG. 39);

FIGS. 40 and 41 are section views of a flow control device using rolled control elements shown in a closed condition (FIG. 40) and an open condition (FIG. 41);

FIGS. 42A-42F illustrate a method of implanting a flow control device into a male urethra;

FIGS. 43A and 43B illustrate a method of disengaging a fixation elements from the sidewall of a body lumen;

FIG. 44 is a perspective view of a flow control device have fixation elements that engage with a body lumen by rotating the device;

FIG. 45 illustrates a flow control device with a plurality of small tines to engage with a body lumen wall;

FIG. 46 illustrates a flow control device with a plurality of small hooks to engage with a body lumen wall;

FIG. 47 illustrates a view of a flow control device in the female urethra anchored using a magnet outside of the urethra;

FIG. 48 illustrates a perspective view of a flow control device having coil shaped fixation elements;

FIG. 49 illustrates a perspective view of a flow control device anchored in a body lumen using a balloon.

### DETAILED DESCRIPTION OF THE INVENTION

Based on these observations and the clinical need, we have developed a novel valve mechanism to allow a person with incontinence to effectively regain control. In one embodiment, there is provided a time-delayed, physiologic, patient activated valve system to control the movement of substances within the body. The valve system is positioned within a bodily lumen such that all of the flow within the bodily lumen flow through the lumen of the device. In another embodiment, the device is activated using an external device or control including a wireless control.

Though the device could be used in any situation in which a liquid, solid, or gas is held in one space and then allowed to move to another, in one embodiment of the invention, the valve is designed to recreate a controllable sphincter similar in function to that of the native urinary system to aid in the treatment of urinary incontinence. This will be used as an example of one use of the valve but should not be taken as the only usage of the device or a limiting embodiment of the invention.

The invention can be composed of any form that allows for a delayed opening of the valve. For urinary stress incontinence, this delayed opening characteristic will maintain the valve in a closed position over the relatively short time frame of the stresses associated with unwanted opening of the native sphincter or contraction of the lumen. Exemplary stresses include times of coughing, sneezing, strenuous activity, and the like typically having a duration normally lasting approximately one second to three seconds.

The valve will however open when a continuous pressure of sufficient magnitude and duration is maintained. The operating characteristics of the device may be tailored so that the amount of pressure and duration of pressure and other factors are set to a level determined by an individual patient. Individual patient incontinence parameters may be determined using patient feedback, testing such as urodynamic tests or other indications of body lumen pressures and characteristics. In the example of urinary incontinence, intraurethral pressure or leak point pressure may be measured with a patient under various conditions of strain (i.e., bearing down) and bladder volume/pressure to customize device response to patient specific values. For example, once a valve device is set to respond to patient specific characteristics, the individual may perform any suitable physiologic technique to increase intralumenal pressure for a period of time sufficient to open the valve or actuate the device. In one exemplary example, the urethra pressure anatomically proximal to the device is increased and sustained for 5 seconds. This time period is exemplary and may be changed to meet individual patient preferences.

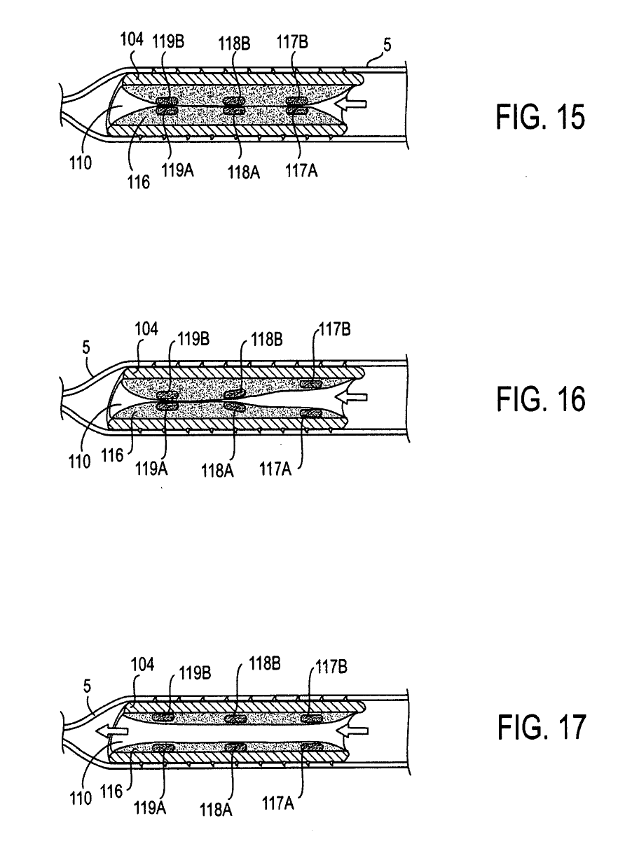

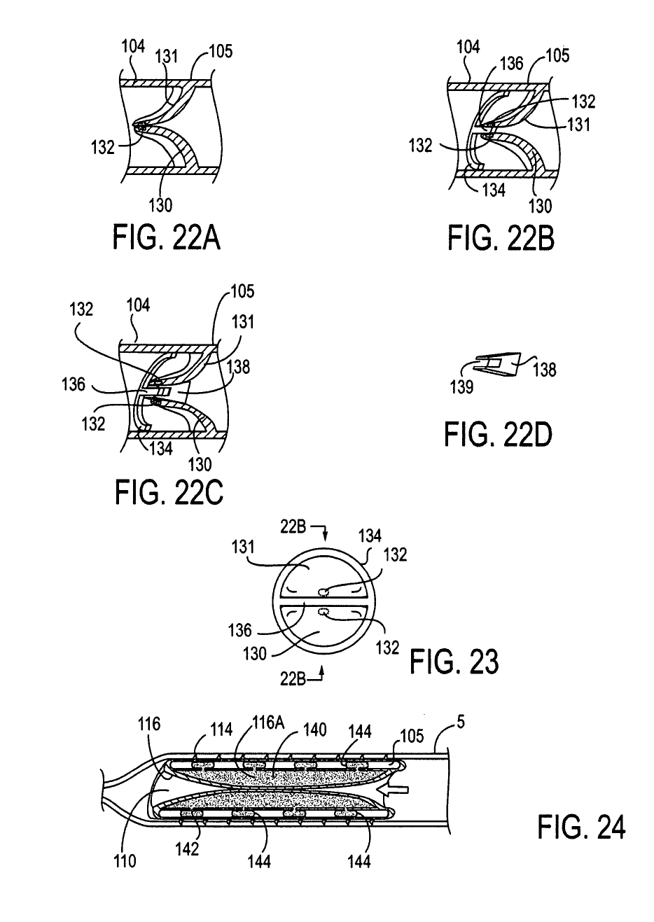

In one embodiment, this mechanism is accomplished through a series (one or more) of individual valves within the device. A valve as used herein may be a physical valve such as shown in FIG. 32 and FIGS. 33A-34B or a structure acting as a valve such as in FIGS. 22A-22C, 26, 28, 30, 36, 37, and 38. In other embodiments, the valve function is performed by closing off the lumen of the device by collapsing the lumen as in FIGS. 5, 9, 12, 21, 40 or closing off a liner within the device lumen as in FIGS. 15, 19, and 24. These and other devices and techniques for closing and opening a device lumen to restore continence in a bodily lumen will be further described in the illustrative embodiments that follow.

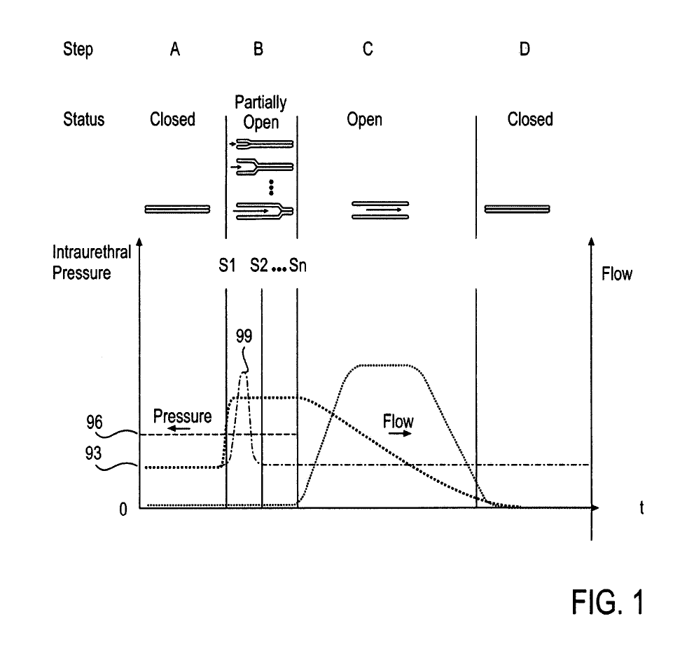

In one aspect of the invention, the valve, structure or valve function response of the device is opened after a prolonged period of increased intraluminal pressure anatomically proximal to the device (i.e., by performing a Valsalva maneuver or bearing down). However, once the device lumen is open, the valve or valves require minimal pressure to remain open. FIG. 1 illustrates an exemplary response profile for the operation of an exemplary device according to one embodiment of the invention.

Using urinary incontinence as an example, FIG. 1 relates intraluminal pressure anatomically proximal to the device and urine flow in an illustrative physiologically controlled incontinence device implanted in the urethra of a person as described herein. Step A shows when the obstructive elements (stages or series of valves) are completely closed when the patient is not bearing down or experiencing stress. Step A shows there is some pressure 93 from a partially filled bladder for example. The pressure 93 is not sufficient to open the valve. The threshold pressure 96 is that pressure above which the device will open. The duration of time for steps S1-Sn is the prolonged duration needed for a pressure at or greater than the threshold pressure 96 needed for the device to open fully. A transient stress 99 illustrates how the device may partially open but the stress 99 is not of sufficient duration or magnitude to open the valve. Step B shows initiation of increased pressure where the first stage (S1) opens. With sustained pressure, the 2^{nd} stage (S2) opens but no urine flow is achieved. Spikes of increased pressure caused by sneezing, coughing, or exertion may open the first two stages S1 and S2 but this temporary pressure transient will not result in urine flow. With continued sustained pressure, the 3^{rd} stage (S3) or final stage (Sn) is open and urine flow is achieved. As shown in Step C, there is little pressure requirement to keep the valves in the stages S1-Sn open due to the optimized operating characteristics of the device. Decreasing urine flow decreases the pressure allowing the closure features of the device to engage and close the device lumen as shown in Step D. The valves close shut to create continence. Consider the specific example where magnets are used to keep the device lumen closed. Once the magnetic field is broken and the magnets in a stage are separated (Step B), then the force needed to keep them separated is low since the magnetic field decreases as magnet spacing increases. Then, as flow decreases through the device lumen (Step C), the magnetic field will increase until the magnets are coupled and the device lumen again closed (Step D). The number of stages, opening pressure of each stage, and orientation of a stage between adjacent stages, within the device body and lumen may be adjusted and customized to accommodate patient preferences or needs. Additional magnet actuation details and other details are provided in U.S. Pat. No. 5,041,092 titled “Urethral Indwelling Catheter With Magnetically Controlled Drainage Valve and Method” and U.S. Pat. No. 6,623,421 titled “External Magnetic Actuation Valve for Intraurethral Artificial Urinary Sphincter” each of which is incorporated herein by reference in its entirely.

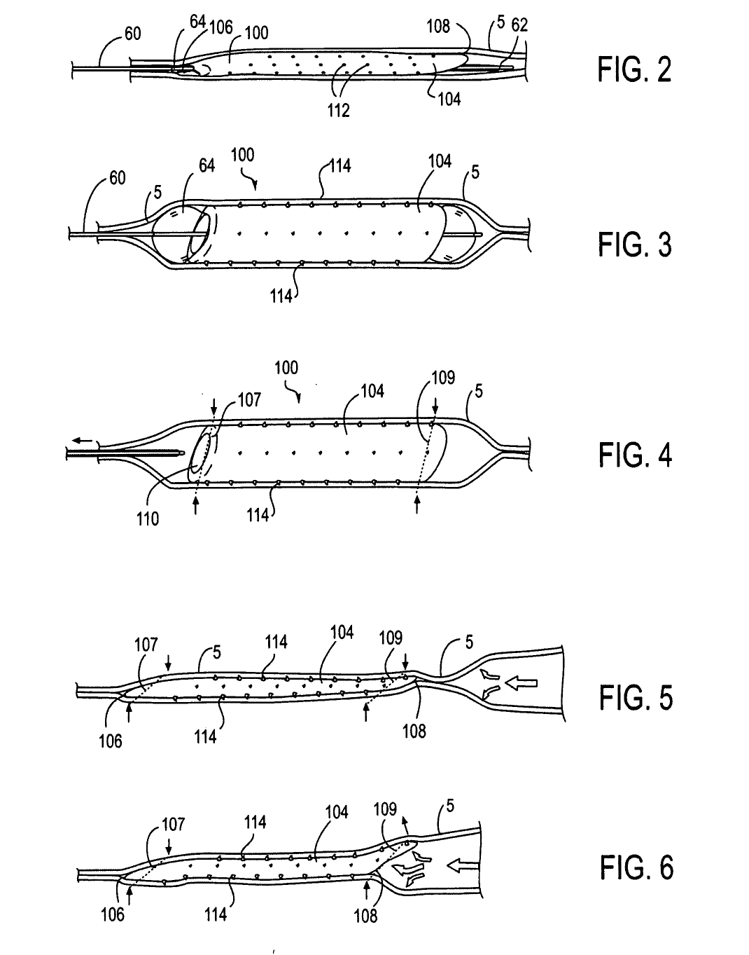

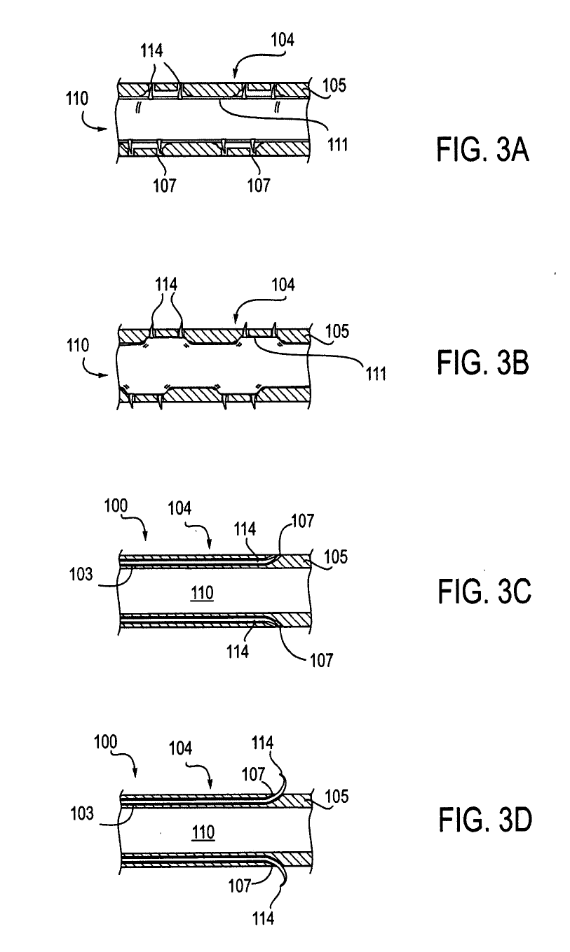

FIG. 2 illustrates an exemplary continence device 100 in a bodily lumen 5. The device 100 is positioned on a catheter 60 and advanced to the desired implant location. The device 100 has a device body 104 with a proximal end 106 and a distal end 108. A device lumen (not visible in the illustrated closed position) extends from the proximal end to the distal end. The sidewall 105 has a number of apertures 112 available for allowing fixation or anchoring elements 114 to pass through the sidewall 105 and into the adjacent bodily lumen 5 as shown in FIG. 3. In FIG. 2, the device 100 is shown in a stowed condition. In FIG.3 the device 100 transitions from a stowed to a deployed and implanted condition through the inflation of the balloons 62 and 64 on catheter 60. Also shown in FIG. 3 are the fixation elements 114 inserted into the bodily lumen 5. The device 100 may transition from stowed to deployed configuration using any suitable technique known in the medical device arts for accomplishing this task such as, for example, by disposing the device within a sheath to stow and then withdrawing the sheath to deploy. The device may itself be inflatable or expandable from a stowed unexpanded condition to a deployed expanded or inflated condition. Similarly, the devices 100 may be modified to allow deployment using other techniques from those used to deploy stents and other device into the vasculature.

FIGS. 3A-3D illustrate embodiments of retractable fixation elements 114. FIG. 3B illustrates cavities in the side wall 105 containing fixation elements 114. In this view, the fixation elements 114 extend beyond the sidewall 105 and engage with the surrounding bodily lumen 5 (see FIG. 4). Returning to FIG. 3A, the sidewall 105 has apertures 107 that allow passage of the elements 114. The fixation elements 114 are attached to a substrate 111. When substrate 111 is withdrawn into the device lumen as shown in FIG. 3A, the fixation elements 114 withdraw below the outer edge of the sidewall 105. Substrate 111 may be withdrawn using vacuum or may be attached to a balloon that, when inflated, deflects the substrate 111 outward and then is detached from substrate 111.

FIG. 3C and 3D illustrate a device 100 having fixation elements 114 within a channel 103 formed in the sidewall 105. The channel 103 ends with an aperture 107 in the sidewall 105. FIG. 3 illustrates the fixation elements in a stowed condition within the channel 103. In this condition, the fixation elements 114 are withdrawn below the surface of the sidewall 105 for transport within a bodily lumen 5. FIG. 3D illustrates the fixation elements 114 in an extended or deployed condition. In this configuration, the distal end of the fixation elements 114 extend through aperture 107, beyond the side wall 105 and into the surrounding lumen 5 as shown in FIG. 4. FIGS. 3A-3D illustrate two exemplary techniques for withdrawing the fixation elements below the sidewall 105 and then controllably extending the fixation elements to engage the bodily lumen 5.

As the illustrative embodiments of FIGS. 3A -3D make clear, the fixation members 114 may be disposed within the sidewall 105 or the lumen 110 while the device 100 is being moved into position within the body lumen 5. Then, once in position within the body lumen 5, the fixation devices 114 are deployed to engage with the body lumen 5 to anchor the device 100 in place. Any conventional fixation element deployment technique may be used, such as, for example, spring loaded, pneumatically actuated, temperature activated (such as when shape memory alloy fixation elements 114 are used) or other techniques suited to the type of fixation element used. Moreover, numerous variations of fixation element design, shape and combinations of fixation element designs and shapes are possible. For example, spikes, tines, barbs and the like may be angled perpendicularly, angled inward, or angled outward in reference to the device body 104. The number and position of the fixation elements 114 relative to one another and the body 104 may vary but are used to prevent migration of the valve within the body lumen 105. In the example of a device 100 implanted in the urethra for urinary incontinence treatment, the fixation elements are used to prevent migration upstream or downstream within the urethra and/or into the bladder. Additional fixation and anchoring devices are described below.

Returning to FIG. 3, the device 100 is moved from a stowed condition (FIG. 2) into a deployed condition by inflating the balloons 62, 64 on catheter 60. As described above, the same motion that deploys the device 100 to open the device lumen 110 may also be used to deploy the fixation elements 114 from their stowed condition in or on the sidewall 105 or within the lumen 110.

FIG. 4 illustrates the device 100 implanted within bodily lumen 5. Catheter 60 is being withdrawn from the device 100 and the lumen 5. Another feature of some embodiments of device 100 is the sidewall angle. The sidewall or device angle may be observed using proximal and distal reference points 107, 109. The proximal and distal ends 106, 108 do not form right angles and the device body 104 is not a right cylinder as best seen in the section view of FIG. 10. The end view of FIG. 11 shows that the device lumen 110 is open when the device is in the deployed condition. While some embodiments may have more cylindrical shapes, the illustrated embodiment is more oblong and would look more like a parallelogram in section view (see FIG. 10 for example). The angled ends of the body allow the device to collapse to conform to the natural collapsing feature of the bodily lumen 5. As shown in FIG. 12, the device 100 collapses to a compact profile within the bodily lumen 5. The end view 13 shows that in this condition the device lumen 110 is closed. Exemplary bodily lumens 5 that naturally partially or completely collapse when not in use are veins, the urethra, the esophagus as well as other portions of the alimentary canal such as the small and large intestines, and the colon. Embodiments of the device of the present invention may be adapted and configured to collapse in harmony with the natural collapse of the bodily lumen into which the device 100 is implanted.

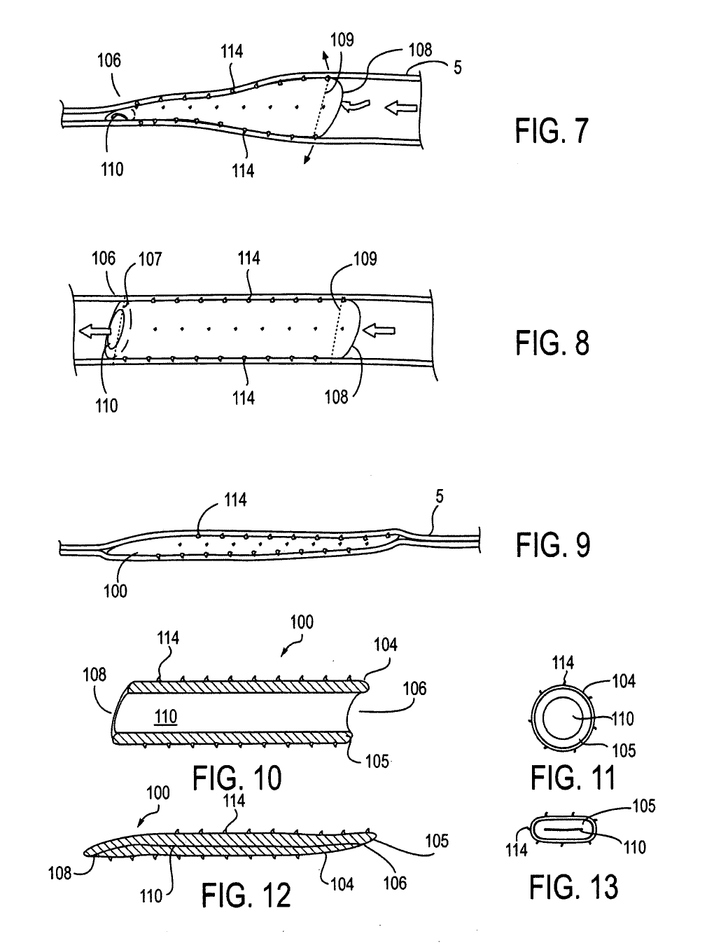

The embodiment of FIG. 4 also illustrates how the device body 104 may be designed to act as a valve. The angled ends tend to leave the device 100 in a closed state. The characteristics of the material used in the body (thickness, elasticity, durometer and other material characteristics) may be modified to give the body 104 the response characteristics described above with regard to FIG. 1. For example, the sidewall thickness may vary along the length of the body 104 such that the various stages of opening are imitated. Similarly, the sidewall thickness may be uniform but the material properties of the body material altered so that different response zones are created again to replicate the staged operation described in FIG. 1. FIG. 5 illustrates FIG. 1 step A. There is some pressure in the lumen 5 but it is insufficient to open the lumen 110. FIG. 6 illustrates the beginning of FIG. 1 step B where the pressure is increased within lumen 5 and the device distal end 108 begins to open (S1). Note the movement of the reference line 109 as the distal end of the body begins to open. FIG. 7 illustrates more intermediate action in FIG. 1 step B where more proximal portions of the body 104 are moving to an open position. FIG. 8 illustrates the fully open device as in FIG. 1 step C. Here, the pressure and flow within the bodily lumen 5 are sufficient to overcome the designed in device bias described above to maintain a closed device lumen 110. FIG. 9 illustrates the closure of the device once pressure decreases as in FIG. 1 step D. This figure illustrates the device in a collapsed condition to conform to the natural collapsing feature of the bodily lumen 5.

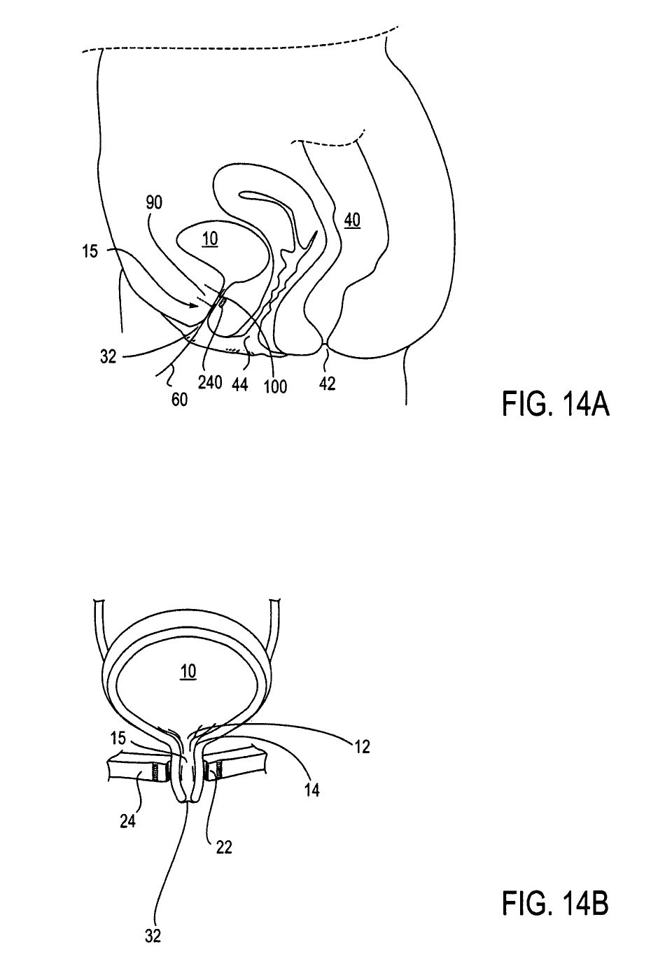

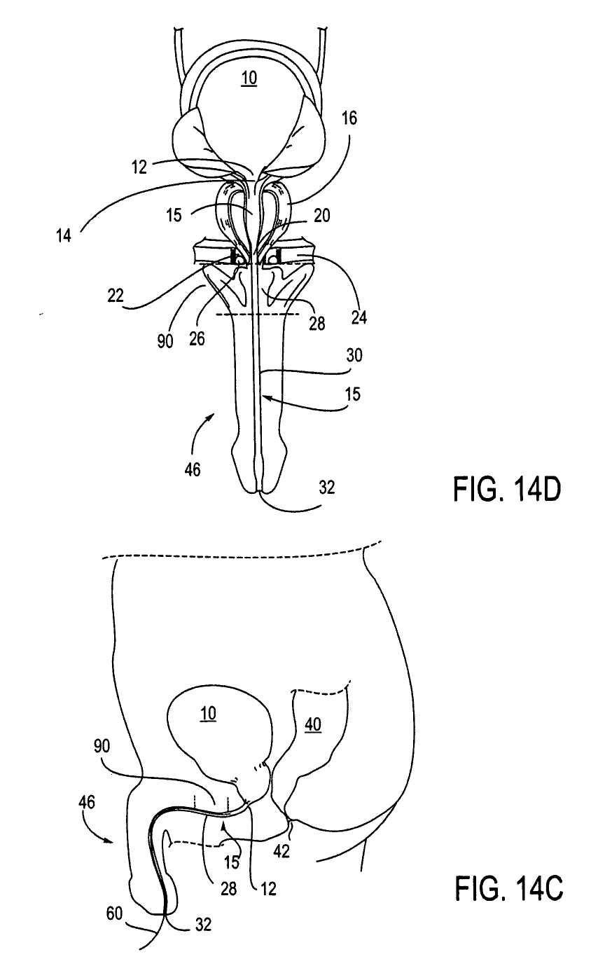

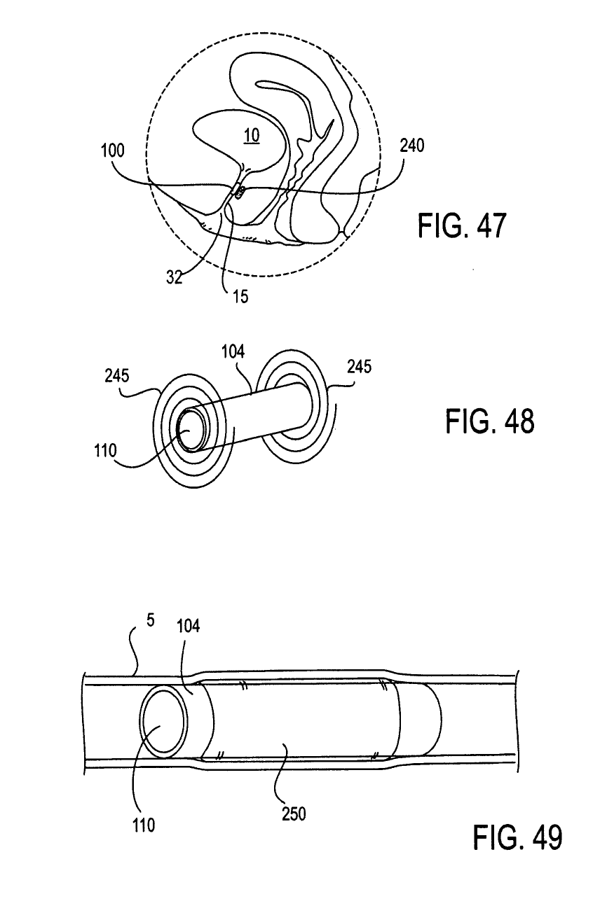

In one specific example, embodiments of the devices described herein may be used to treat urinary incontinence. The anatomy of the male and female urethra 15 will be described with reference to FIGS. 14A-14D. FIGS. 14 A and 14B illustrate sagittal and coronal plane views, respectively, of the human female abdomen. In the female, the urethra 15 extends from the bladder 10 to the external urethral orifice 32. The view in FIG. 14A illustrates the external urethra orifice 32 relative to the vagina 44, the anus 42 and colon 40. Also shown in FIG. 14A is the device implantation region 90 where device 100 will be implanted into the urethra 15. In the female, one exemplary device implantation region 90 is mid-urethra. An embodiment of the device 100 is illustrated on the catheter 60 and inserted into the implantation region 90. This illustration also shows the placement of the magnetic anchoring device 240 described below with regard to FIG. 47. FIG. 14B illustrates a coronal view of the female abdomen. The female urethra 15 extends from the bladder 10 to the external urethral orifice 32. The flow path from the bladder 10 to the external urethral orifice 32 is as follows: bladder 10 through the bladder neck 12 and the internal urethral sphincter 14 into the urethra 15. Next, the flow is through the external urethral sphincter 22 and the urogenital diaphragm 24 and out through the external urethral orifice 32. In females, the external sphincter 22 is at the same level as the urogenital diaphragm 24. FIGS. 14C and 14D illustrate sagittal and coronal plane views, respectively, of the human male abdomen. In the male, the urethra 15 is longer than the female and extends from the bladder 10 to the external urethral orifice 32 and the top of the penis 46. The view in FIG. 14C illustrates the external urethra orifice 32 relative to the penis 46, the anus 42 and colon 40. Also shown in FIG. 14C is the device implantation region 90 where device 100 will be implanted into the male urethra 15. In the male, one exemplary device implantation region 90 is the bulbous urethra. FIG. 14D illustrates a coronal view of the male abdomen and penis. The male urethra 15 extends from the bladder 10 to the external urethral orifice 32 at the tip of penis 46. The male flow path from the bladder 10 to the external urethral orifice 32 is as follows: bladder 10, bladder neck 12, internal urethral sphincter 14 into the urethra 15. Next, flow passes the prostate gland 16 through the prostatic urethra 18 and then flow passes through the membranous urethra 20 and passes the external urethral sphincter 22, the urogenital diaphragm 24 and the bulbourethral gland and duct 26. Then, the flow passes through the bulbar urethra or bulbous urethra 28 passing through the spongy/penile urethra 30 and out through the external urethral orifice 32 at the end of penis 46. In males, the external sphincter 22, membranous urethra 20, urogenital diaphragm 24 and the bulbourethral glands 26 are all at the same level of the urethra 15. The bulbous urethra 28 extends from the end of the urogenital diaphragm 24 to the beginning of the penile urethra 30.

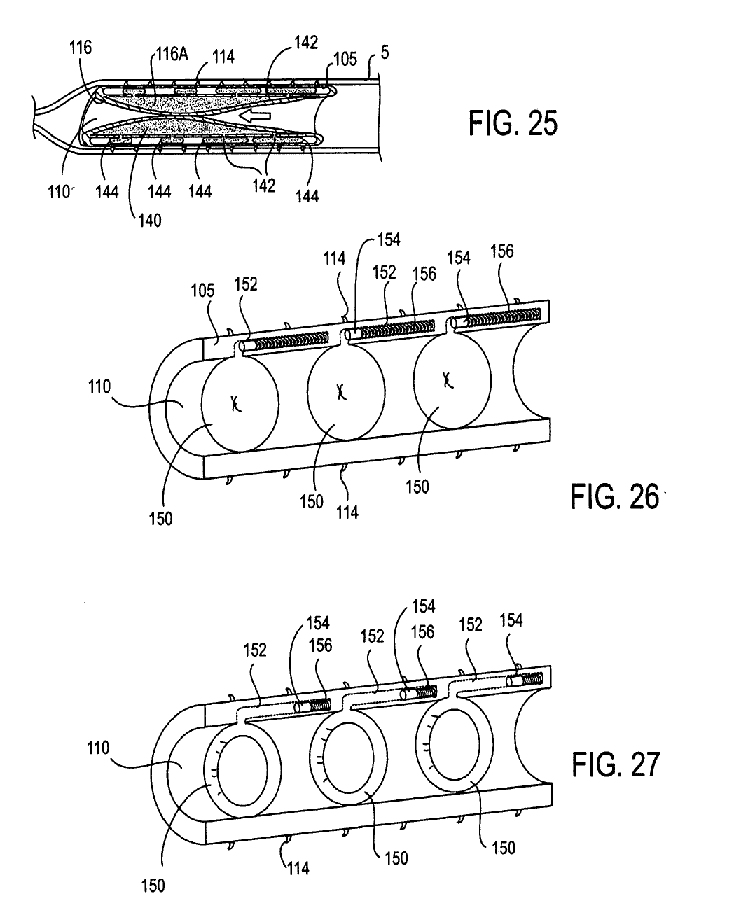

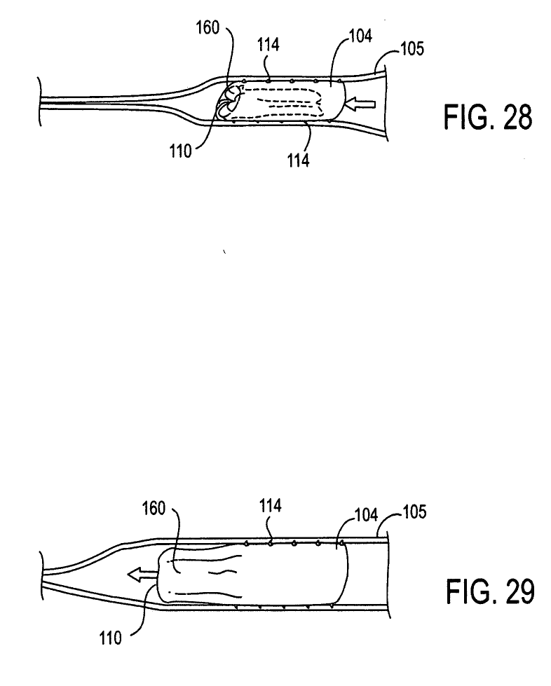

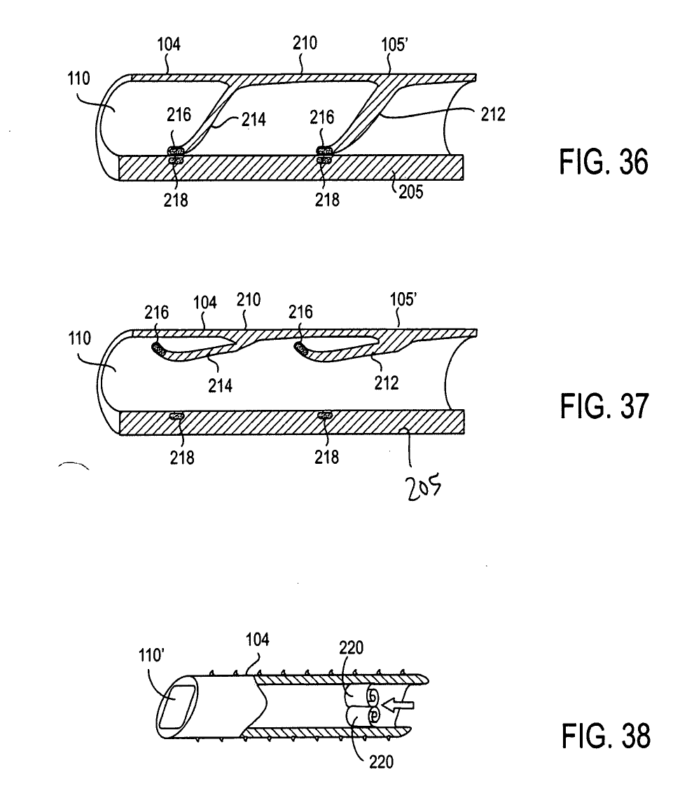

In some embodiments the invention is configured for use as an intra-urethral device. In this type of embodiment, the device body 104 may have a generally cylindrical body with a lumen 110. The body 104 is adapted and configured to fit within the urethra 15 so that all flow through the urethra 15 flows through the lumen 110. The body 104 and the components therein may be aligned to facilitate collapse of the device under the natural pressure of the urethra 15 to close then not in use. Internal components may also be angled to reduce the force needed for the device 100 to collapse when not in use. Examples of angled components include angled ends 106, 108 as in FIGS. 2-13 or angled interior components as in, for example, valves 170 in FIGS. 32 or flaps in FIG. 22 or other components as illustrated in FIGS. 36, 28, and 30. The body 104 is made from a flexible biocompatible material selected for biomechanical compliance with the body lumen 5 such as the urethra 15. Generally, the body 104 is longer than it is wide so that it will maintain an implanted orientation when in the bodily lumen. In one embodiment intended for use in the urethra 15, the device 100 will about 1-2 cm in length. General size ranges for use in humans range from 1-4 cm in length and 0.5-1.0 cm in diameter. The diameter of the human urethra is dynamic based on urethral pressure, elasticity of the mucosa among other factors. This dynamic range is accommodated by the various design features described herein to accommodate a dynamic device size by including features that aid in collapsing the device 100 when not in use. The urethral diameter may range from 5 mm to about 10 mm. In order to maintain or optimize biomechanical compliance, some embodiments of the body 104 are adapted to decrease in diameter as the urethral walls collapse as pressure and urine flow decrease. The walls of the urethra 15 naturally collapse and the lumen flattens when urethral pressure is removed. The device 100 has a hollow, elongate body 104 with a lumen 110. In some embodiments, a liner 116 is disposed within the device lumen 110. As will be described in the embodiments that follow, the liner 116 is moveable from a normally closed position to an open position. In the closed position, the device lumen 110 is blocked. In the open position, the device lumen 110 is open. Exemplary materials for body 104 and liner 116 include silicone and other elastic biocompatible polymers. The body lumen 110 may be uniform along the longitudinal axis of the device 100. This would mean that the thickness of sidewall 105 is constant or nearly so such that the device lumen 110 extends generally centrally along the longitudinal axis of the body 104. Alternatively, as best seen in FIGS. 36 and 37, the device lumen 110 may be offset. The offset lumen leaves an amount of contiguous space on the thicker sidewall side 205. The added space in the sidewall 205 may be used for storage of other elements used in the device, such as electronics for monitoring, recording or controlling the operation of the device. Alternatively, the space may be used for other components of the device such as fluid diaphragms (see for example FIGS. 24, 25) or for pistons (see for example FIGS. 26 and 27). Other device elements include, but are not limited to, electronics, storage devices, and other components to facilitate operation based on the particular operating mode of the device. In another alternative, the device lumen 110 follows a non-linear pathway along the length of the body 104. The non-linear pathway may be formed by thicker and thinner sidewall portions or by adding baffles to the interior lumen wall. Alternatively, a non-linear flow path may be provided by using flow control devices that open in different orientations. For example, FIG. 32 illustrates a plurality of flap valves 170. The location of the hinges 174 may be moved to different locations rather than having the hinges 174 at the top, middle of the lumen as illustrated. In another alternative, the flow device may be a rolled controlled flow blocking element 224 as illustrated in FIGS. 40 and 41. The manner in which the elements 224 unroll may also be used to produce a non-linear flow through the device lumen 110.

In some embodiments, a deformable liner 116 lies within the device lumen 110. Deformation of the liner 116 closes off flow through the device lumen 110. The liner 116 may be two or more pieces or a single piece. The liner 116 may have any shape so long as it closes off the device lumen 110. In the embodiments that follow the liner 116 is closed using a number of different techniques. The liner 116 may be closed off using magnet pairs placed on opposite sides of the device lumen 110. The magnets hold the lumen in the normally closed position by pinching down horizontally to close off the liner 116 thereby closing off the device lumen 110. When magnet pairs are described herein, that includes the use of two magnets with attractive poles or one magnet and a ferrous material. The magnet pairs may be permanent magnets or temporary magnets such as electromagnets. In the embodiments that follow, increased bladder pressure overcomes magnetic force of the magnet pair, opening the device lumen 110 and allowing the bladder 10 to empty. Magnets may also be configured differently other than in pairs. For example, two or more magnets may be joined to a single magnetic or ferrous material.

FIGS. 15, 16 and 17 depict one embodiment where the device 100 is an elongate, generally cylindrical body 104 that fits within the urethra 15 so that all flow through the urethra flows through the device body lumen 110. A deformable liner 116 lies within the device lumen 110. The liner 116 is closed off using a number of magnet pairs 117A/B, 118A/B and 119A/B. The magnet pairs are aligned horizontally to close off the deformable liner 116 to thereby close the device lumen 110 to create continence. FIG. 15 depicts step A of FIG. 1 where all stages or magnetic pairs are closed and proximal intraurethral pressure is minimal. FIG. 16 illustrates the beginning of step B where an increase in pressure has overcome the magnetic force of the magnetic pair 117A/B and the first stage opens. If pressure is sustained, then the second magnetic pair 118A/B will open but urine does not flow. If pressure continues to be sustained, the third magnetic pair 119A/B opens and urine flows as shown in FIG. 17 and corresponding to step C in FIG. 1. At this point, a minimal urethral pressure or urine flow is required to keep the stages or magnetic pairs open. The number of stages or magnetic pairs and opening pressure of each magnetic pair or stage can be adjusted and customized to fit individual patient requirements or preferences. The magnet pair may have any of a number of different cross-sectional shapes to achieve coaption of the deformable liner 116. Exemplary magnet pair shapes include round, polygonal, at least one flat side—where flat sides are drawn together as well as complementary shapes such as male-female shapes that interlock when engaged.

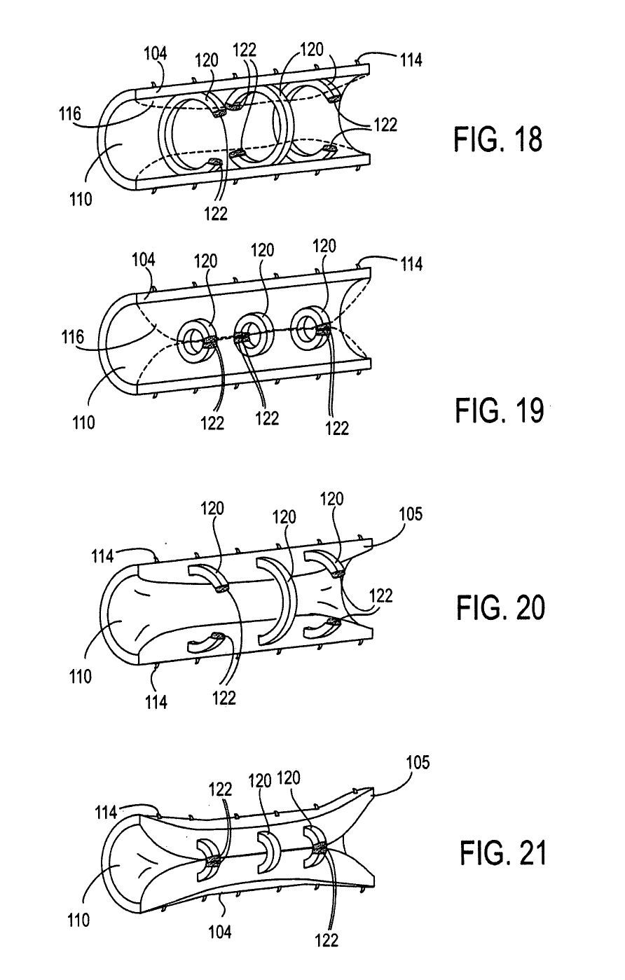

Additional magnetic closure devices are illustrated in FIGS. 18-21 that utilize radial cuff magnets having rings 120 and magnetic ends or pairs 122. The cuffs are aligned in series along the longitudinal axis of the liner 116. These devices impart force resulting in radial closure of the liner 116 to shut off device lumen 110. FIGS. 18-21 are similar to FIGS. 15 and 17 except the stages are composed of magnetic cuffs that deform the liner to close the internal lumen. FIG. 18 and 19 illustrate a magnetic radial cuff 120 within the liner 116. FIG. 19 corresponds to stage A in FIG. 1 where all of magnetic pairs are together and the liner 116 closes off the lumen 110. FIG. 18 illustrates stage C of FIG. 1 where internal pressure has opened the cuff and the lumen 110 is unobstructed. FIGS. 20 and 21 illustrate an alternative embodiment that does no use a liner 116 but instead uses the magnetic cuffs 120 to collapse side wall 105 to close off the lumen 116. FIG. 20 corresponds to stage C in FIG. 1 where the device lumen 110 is unobstructed. FIG. 21 corresponds to the stages A or D in FIG. 1 where the device lumen 116 is closed.

In some embodiments, the magnets or magnetic cuffs are arranged within a sliding track so that when they attract each other, they move together along the track. The opposite ends or magnet pairs are connected to each other via the cuff or ring. The attractive force of the magnet pair draws the ends of the cuff together to constrict and close off the device lumen 116. Alternatively, the track may include a line of magnets of decreasing effective strength to ensure that when the device is open, the cuff ends are not overextended into an inoperable position. The length of cuff movement may be controlled by mechanical stops or by placing an opposing magnet at the point of maximum cuff travel. As the magnetic pairs approach the opposing magnet at the maximum point of travel, the repulsive force will act as a biasing force to aid in returning the device 100 to a normally closed position.

In another alternative embodiment, the magnetic force within magnetic pairs is variable. Magnetic force variability may help reduce the likelihood that magnets will come into contact and not separate. Magnetic variability will also be a useful factor is adjusting device characteristics or to match patient parameters or preferences. For example, a patient's urodynamic or leak point pressure testing results may be used to determine individual urethral pressure and suitable magnetic hold force preferences for an individual.

FIG. 22A illustrates another alternative device 100 having magnetic closure flaps 131, 130. The thickness of the flaps 130, 131 may be adjusted to control how resistive the flaps are to flow pressure. Additionally, the magnetic coupling force in the magnet pairs 132 may also be adjusted. For example, different size or strength magnets may be used within a device lumen 110 or different materials may be used for flaps 131, 130 or the dimensions of the flaps 131, 130 may be altered to change the pressure response of a flap or combinations of any of the above. FIG. 22B and 23 illustrate the use of a spacing frame to alter the magnetic coupling force between magnets 132. The spacing frame 134 fits within the device lumen 110 along the sidewall 105. A spacing bar insert 136 fits between and adjusts the magnetic field and hold force of the magnets 132. As shown in end view of FIG. 23, the spacing bar insert 136 prevents the magnets 132 from making direct contact and also reduces the hold force between them. Since the magnets are typically manufactured as integral components of the device component where used, the spacing frame 134—with various width bars. 136 provides a user greater control of pressures used for opening the device lumen 110. Additional variability may be provided by an insert 138. The insert 138 has an opening 139 adapted to attach to the bar 136. These embodiments illustrate how magnetic contact area could be adjusted by insertion of a spacer. In another alternative, the insert or the bar 136 may be a hollow spacer may be used to set a basic spacing. Thereafter, the hollow spacer may be filled with materials having various qualities to provide additional adjustment of the magnetic characteristics of the device. One exemplary material is a μ-material.

In another alternative embodiment, the liner 116 is filled with a fluid or material 140 that moves in the desired pressure range. Variations of hydraulic pistons coupled to liners could be used to control opening of the device lumen 110. Increased pressure against the liner 116 pushes fluid 140 from the liner 116 into the piston or an expandable chamber. Piston is filled with viscous material or a biasing element that must be overcome to open the lumen or the movement of the fluid itself must be overcome to allow the device lumen 116 to open. FIG. 24 depicts an embodiment where the deformable liner 116 is coapted with viscous fluid 140 that also fills cuffs or diaphragms 144 in the sidewall 105. When urethral pressure anatomically proximal to the device is increased, the pressure forces the viscous fluid 140 out of the liner interior 116A, through apertures 142 into the expandable diaphragm 144. This is similar to the beginning of step B in FIG. 1. If there is sufficient sustained pressure as shown in FIG. 25, the following stages or expandable diaphragms 144 will also expand and more of the fluid 140 is urged from the liner interior 116A and into the sidewall in the expandable diaphragms 144. Once sufficient fluid 140 has been moved and the volume of 116A reduced, then the device lumen 110 will open to allow passage of urine. Alternatively, there could just be one stage or all stages could be attached to one piston. Once urine flow has stopped or diminished the force of the expanded diaphragms 144 will drive the viscous fluid 140 back into the cuffs or interior liner 116A and create continence. FIGS. 26 and 27 illustrate another alternative embodiment where the interior of the expandable annulus or cuff 150 is in communication with a piston cavity 152 formed in the sidewall 105. The piston head 154 is coupled to a biasing element 156. FIG. 26 illustrates this embodiment in stages A or D of FIG. 1. FIG. 27 illustrates stage C of FIG. 1. In operation, increasing pressure in the device lumen 110 pushes the fluid 140 inside the diaphragm out of the annulus and into the piston chamber 152. The fluid 140 pushes the piston head 154 and compresses the bias element 156. The bias element may be a spring or other conventional mechanism to provide a bias force to eject the fluid from the piston cavity 152 when the lumen pressure decreases. The viscous fluid 140 used in this application could be hydrogel, oil, gel or any suitable biocompatible material with viscosity and compressibility characteristics suited to operating in the pressure ranges used within bodily lumens. The viscosity of the viscous fluid 140 may also be altered dynamically to adjust overall device pressure response and other characteristics such as opening threshold pressure and duration.

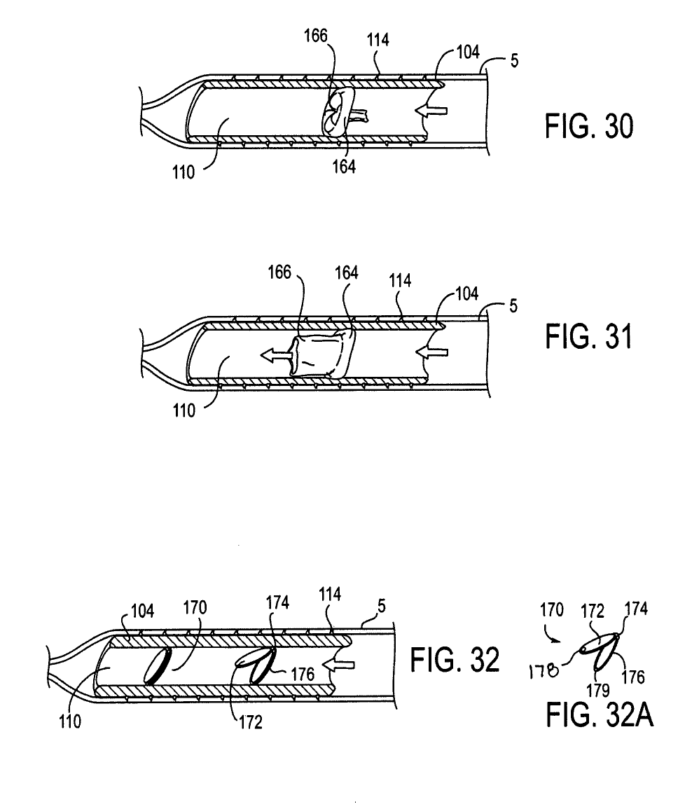

As illustrated in several of the embodiments described herein, embodiments of the invention do not require conventional valves but may instead rely on material properties of materials within the device lumen 110 to achieve the same effect of opening a passageway after a defined period of pressure is maintained. FIGS. 28 and 29 illustrate a a cylindrical, everting balloon 160 within the device body 104. The balloon 160 contains a viscous substance such as oil or other materials with viscosity and other properties suited for use in the pressure ranges used by bodily lumens used with the device. FIG. 28 illustrated the balloon in a closed condition. When lumen pressure is applied, the balloon 160 distends lengthwise. The material within the balloon moves under the force of luminal pressure to stretch the balloon. As the balloon 160 stretches, the luminal diameter and the length of the balloon increase as shown in FIG. 29. Once the lumen finally opens completely due to prolonged pressure, the lumen will stay open with minimal pressure applied. Similar to other embodiments, the closing force will be adjusted by tailoring the pressure response of the material used to fill the balloon 160 as well as the material selected and design parameters of the balloon. In another alternative embodiment, an everting structure 166 may be mounted within a frame 164 within the device lumen 110. The everting structure 166 operates similar to the eversion balloon of FIGS. 28 and 29, as lumen pressure increases the eversion structure 166 unrolls from the frame until the lumen 110 is open. Once lumen pressure decreases, the everting structure will roll up into the annulus 164 and return to the closed position shown in FIG. 30.

In another alternative embodiment, the liner 116 is partially or completely filled with a material to close off the lumen. Similar to the above embodiments, the material would have characteristics selected to be operable in the pressure ranges used for bodily lumens. In one embodiment, the liner is partially or completely filled with one or more materials that maintain the liner in a closed position. The type of material in the liner determines how the liner moves from the closed to the open position. Movement of the liner to the open position is governed by the pressure response characteristic of the material. In one specific example, a hydrogel is used to partially or completely fill the liner 116. One representative hydrogel is polyethylene glycol (NOF Corp. Tokyo, Japan). In another embodiment, there is an electrorheologic material in the liner. One representative electrorheologic material is Verroflo ER100 available from Lord Corp, in Cary, N.C. In another embodiment, there is a magnetorheologic material in the liner 116. One exemplary magnetorheologic material is MRF-241ES manufactures by Lord Corp. of Cary, N.C. In yet another alternative embodiment, the liner 116 is filed with “memory foam” or “open cell foam.” The material in liner may be modified prior to or after implantation based on patient specific criteria. Exposure of the material to a material appropriate stimulus (i.e., magnetic field to a magnetorheological material and the like) RF, magnetic field, temperature, etc. could adjust the operational characteristics of the device—more urethra pressure to urinate, less pressure to urinate, disable the device (body lumen is as unobstructed as permitted by the device or completely open in the case of a valve) or any other adjustment needed to modify the device performance characteristics.

These material embodiments operate generally in the following manner. As proximal intraurethral pressure increases, the hydrogel will deform to allow passage of urine. The hydrogel may also change viscosity in response to a stimulus including a change in temperature, electrical stimulation, chemical stimulation, or other stimulation such as ultrasound. Electrorheologic material could change viscosity within an electric field generated by a battery or by wireless control. Magnetorheologic fluid could change viscosity in response to a magnetic field generated by a magnet, electromagnet, or permanent magnet or wireless control.

Turning now to FIG. 32 and 32A. This illustrative embodiment uses flap valves 170 within the device lumen 110 to control flow within the bodily lumen 5. Valves 170 are angled to lay better within a collapsed urethra as described above. Each flap valve 170 includes a cover 172 with a hinged attachment 174 to a seat or rim 176. The seat 176 may be on or within the sidewall 105. The cover 172 is positioned to close off the device lumen 110. The hinged attachment 174 may also be biased using a spring or other suitable biasing element to close the cover 172 once pressure drops off as in stage D of FIG. 1. While the hinged attachment provides adjustability for closing and opening force for the cover 172, a magnetic pair 178/179 may also be used to adjust the pressure response of the valve 170. Each individual valve 170 is composed of a disc or rim 176 that has some magnetic properties (for example being made of metal, being a magnetic disc itself, having a magnet placed on the disc, etc) that is seated to a lip of the cover 172 that also has magnetic properties (for example being made of metal, being a magnetic disc itself, having a magnet placed on the disc, etc). The magnetic coupling pair on the disc and the lip provide a relatively strong force to maintain the valve in a closed position. The magnetic force may also be adjusted as described herein. But like other magnet embodiments described herein, once opened, the magnetic closing force will rapidly decrease as the distance between the magnet pair increases. Further, the movement of the substance (whether liquid, solid, or gas) through the valve 170 will also aid in maintaining the valve in an open position. In other embodiments, the flap valves 170 are biased such that they will begin to close as urethra pressure decreases after urination. In another embodiment, the valve has a rebounding mechanism to maintain it in a closed position juxtaposed to the lip. This rebounding mechanism can be something such as a normal spring, a torsion spring that acts as a hinge, a rubber band or other elastic type material, an oppositely polarized magnet and the like. In other alternative embodiments, the cover 172 may be divided further into two, three or more segmented covers. Each segmented cover has a hinged connector 174. The segmented covers operate together to close or open the device lumen 110. In one embodiment, the segmented covers 172 are adapted and configured to imitate the leaflets of a heart valves.

Another technique to vary the device pressure response to open is the use of a non-linear device lumen. In other words, the device lumen 110 does not follow the longitudinal axis of body but instead some curved or angled pathway. Unlike FIG. 32, the placement of the valves 172 in a non-linear lumen would off set valve hinges 174 rather than in line as illustrated in FIG. 32. Offset valves 172 will alter the fluid dynamic properties of the device lumen flow. Techniques such as this provide further pressure response configurability to the device performance. Another technique to vary the fluid dynamic properties of the device lumen 110 is to use baffles instead of or in combination with valve offsets to adjust the fluid flow path and properties through the device lumen 110.

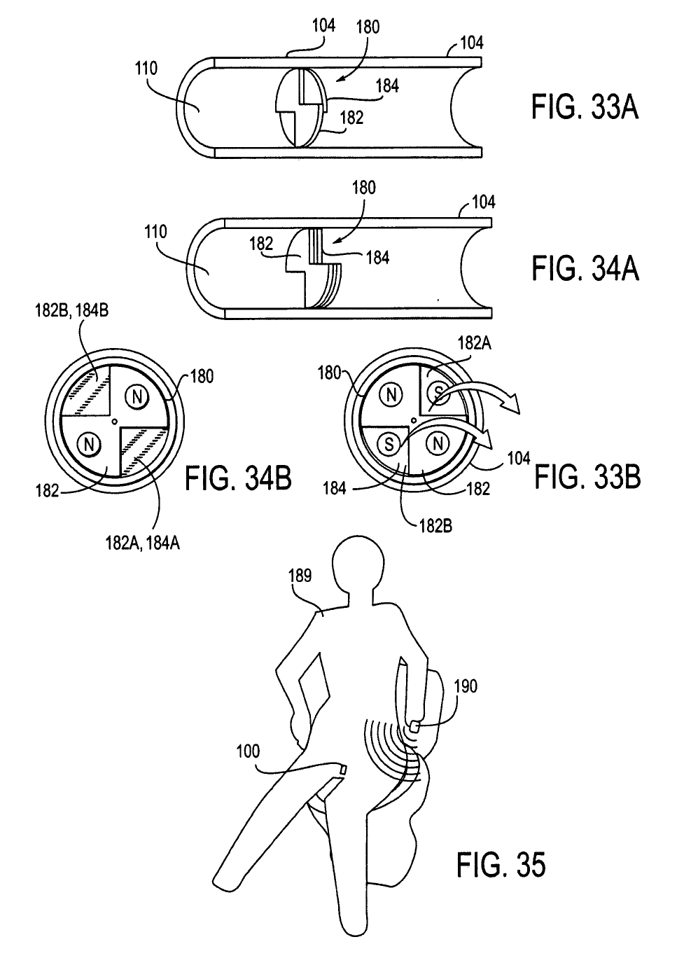

FIG. 33A illustrates yet another alternative embodiment of a device of the present invention. In this embodiment, a magnetically actuated rotary valve 180 is provided within the device 100 to control the flow through the device lumen 110. The rotary valve 180 is opened and closed using an external force. In this embodiment of the invention, there is provided a wirelessly controlled valve which requires no battery for actuation into closed, open or intermediate positions. The valve 180 is adapted and configured to sit within the device lumen 110 on, within or partially within the sidewall 105. There is also provided an external device 190 which generates energy to deliver to the valve 180 using any of a number of methods to actuate the valve 190 and control its position wirelessly into the closed, open or intermediate positions.

In one illustrative embodiment, the device 100 has a body 104 with a cylindrical shape. The ends 106, 108 may form a right cylinder or may be angled to form a type of parallelogram when viewed in cross section as described above. The device lumen 110 contains a plurality of disks 182, 184 at least one of which is free to rotate on an axis. The disks 182, 184 have a plurality of openings 182A,B and 184A,B such that when the rotational disk or disks rotates into certain positions, the alignment of the disks is such that the openings either allow (FIG. 34B) or prevent completely (FIG. 33B) or to a degree the passage of fluid or other materials from one end of the device body 104 to the other when the disk is rotated. The disks 182, 184 contain a plurality of magnets arranged such that when a plurality of external magnetic forces is applied, the disk 182 or 184 will rotate. The fixed disks (one or the other of 182, 184 that does not rotate when exposed to an external force) or other structural members of the body 104 may also have magnets which situated in opposition or in conjunction with the poles of the magnets on the rotational disks 182, 184 such that that disks will return to a predetermined open, closed or intermediate position. Instead of using magnets to return the disk to a predetermined position, a plurality of other methods, including but not restricted to biasing springs, may be used. The external magnetic field will be of such strength so as to overcome the biasing force (magnetic or otherwise) that holds the rotational disk into a predetermined position.

In the embodiment in FIGS. 33A-34B, an external magnet is used to actuate the implanted valve 180 inside the device body 104. In this embodiment, the valve 180 is implemented with two disks 182, 184. One disk is fixed in relation to the device body 104. That disk is placed concentrically inside the device lumen 110. The second disk is connected to the first disk via an axis inserted thru the center of both disks. The second disk is allowed to rotate along that axis. The disks may also be mounted on, in or within the sidewall 105 or otherwise within the lumen 110 without the use of an axis inserted through the disks. There are two or more diametrically opposed openings (circular or other shape) in the disks such that when the openings are aligned, there is a path for fluid flow thru the disks, but when the disks are rotated 90 degrees to one another, the openings do not overlap and there is no path for fluid flow thru the disks (analogous to a salt shaker opening). The second disk will move in response to an external magnet because of the diametrically opposed small rare earth magnets attached to the disk. When a magnet of the similar polarity is placed near the disk, it will rotate to minimize repulsion and so the diametrically placed magnets will move 90 degrees away from the external magnet. The valve is implemented with two disks. One disk is fixed in relation to the device body 104. That disk is placed concentrically inside the cylinder. The second disk is connected to the first disk via an axis inserted thru the center of both disks. The second disk is allowed to rotate along that axis. There are two or more diametrically opposed openings (circular, sector, diamond, or any other shape) in the disks such that when the openings are aligned, there is a path for fluid flow thru the disks, but when the disks are rotated 90 degrees to one another, the openings do not overlap and there is no path for fluid flow thru the disks similar to a salt shaker opening. The second disk will move in response to an external magnet because of the diametrically opposed small rare earth magnets attached to the disk. When a magnet of the similar polarity is placed near the disk, it will rotate to minimize repulsion and so the diametrically placed magnets will move 90 degrees away from the external magnet. The valve, when in the open position, consists of two disks whose openings are aligned as shown in FIG. 34B. One disk is fixed and the other rotates on an axis 16. The disks have magnets. The fixed disk magnet 14 is oriented such that the rotating disks magnet 20 repel each other and so cause the valve to be biased into the closed position (FIGS. 33A and B) when no other forces are present. FIG. 34B shows the device in the open position and the fluid in the device lumen 110. In this configuration, the fluid or other substance in the bodily lumen 5 would flow thru the device 100. In FIG. 33B, the magnets are aligned although they are oriented to repel since the external magnets poles are aligned to overcome the smaller disk magnets' repulsive force.

The disks need not be oriented perpendicularly to the body of the implanted device. The disks could be angled so as to increase the surface area of the openings and to allow for additional compressibility of the valve.

FIG. 35 illustrates an embodiment of a patient 189 sitting on a toilet 191. An embodiment of the device 100 is implanted into the urethra 15 of the patient 191. In one embodiment, the device 100 contains a magnetic valve as in FIGS. 33A-34B that is actuated by an external magnet 190 held by the patient 189 in a specific orientation relative to the valve body 104 and urethra 15. The valve 180 will then open and allow urine to flow outside the body. When the external magnet 190 is removed, the valve 180 closes due to the biasing force, magnetic forces or other forces that are part of the response characteristics of the device 100. When the valve closes, urine flow stops if not before. Wireless control could be used in conjunction with follow up testing and patient specific complaints or urological pressure measurements to modify the device operating characteristics. A wireless device 190 could be used to control the opening and closing of the intraurethral device to allow passage of urine, to control or adjust the opening pressure of the device so that greater pressure or more sustained pressure is needed to open all stages of the device, and could be used to acquire data about the patient. The receiver within the device may have a power storage unit, a control unit, and an actuator or property adjustor. The power storage unit may have a battery or capacitor. The control unit controls the actuator and stores data. The actuator may have a motor or adjust fluid characteristics and will adjust the stages. Additionally, the actuator may control the distance between the magnets and thus control the attractive force between them. Alternatively, it can control the viscosity of the hydrogel and thus alter opening pressure. Patients could use wireless control by increasing or decreasing the opening pressure required for physiologic control. The use of an external control device allows for device operational characteristics to be adjusted without removing the device. If a patient were going to engage in some exercise or other form of exertion, the patient could increase the strength or lever of physiologic pressure needed to open the device lumen 110. Alternatively, if the patient were going to sleep, they could also adjust the strength and operating characteristics as desired. Additional details of the construction and operation of a wireless controller 190 are found in U.S. Pat. No. 6,638,208 titled “Intraurethral Continent Prothesis” which is incorporated herein by reference in its entirely.

In addition to the devices described above, additional alternative closing devices may be used to control or alter the pressure response characteristics of the device 100. FIGS. 36 and 37 illustrate side views of alternative devices having altered sidewalls 105′ and magnetic ligaments 212 and 214. FIG. 36 illustrates the ligaments 212, 214 closed where the magnet pairs 216 and 218 are engaged. FIG. 37 illustrates when the pressure in lumen 110 has overcome the material forces of the ligaments 212, 214 and the magnet pairs 216, 218 and flow through lumen 110 is permitted. The dimensions, materials and/or material properties of the ligaments 212, 214 may be altered to adjust the pressure response of each ligament. For example, ligament 212 is thicker where attached to the sidewall 105′ and tapers down to a thinner section near magnet 216. In contrast, the ligament 214 is thinner overall than ligament 212 and has a nearly uniform thickness along its length. The characteristics and response of the magnet pairs 216 and 218 may be adjusted as described herein. Additionally, the sidewall 105′ has thicker regions 205 and thinner regions 210. In one embodiment, the sidewall thickness is adapted to provide a non-linear lumen 110. In another embodiment illustrated in FIGS. 36 and 37, the sidewall thickness provides a contiguous space for the storage of device components, electronics or other system elements as desired.

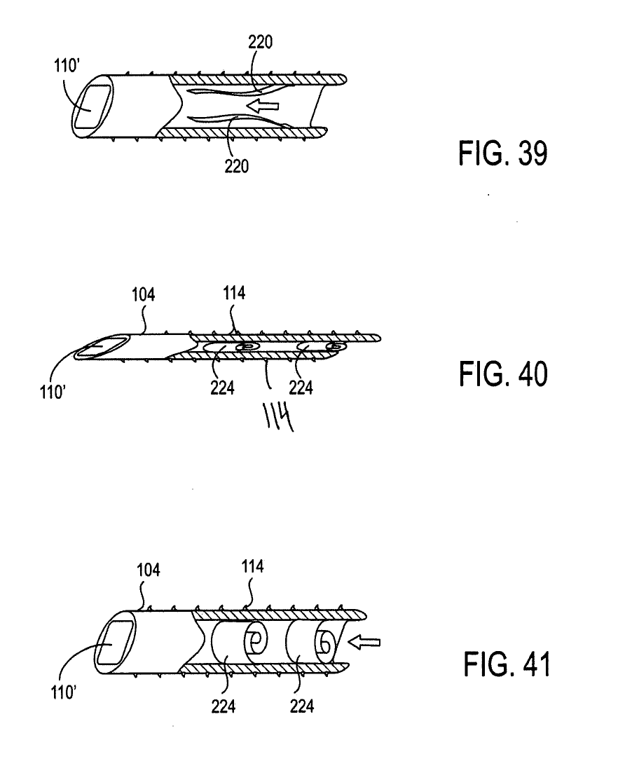

FIGS. 38 and 39 illustrate a non-circular lumen 110 and a pair of rolled controlled blockage elements 220. FIG. 38 illustrates the blockage elements 220 in a closed position. The elements 220 may be formed of any suitable material. The pressure response is determined by the amount of pressure needed to unroll the element 220. FIG. 39 illustrates the blockage elements in an open configuration where the lumen 110 is unobstructed. When pressure drops, the elements 220 return to the rolled configuration and the lumen 110 is closed.

FIGS. 40 and 41 illustrate alternative rolling blockage devices 224. Similar to blocking elements 220, the blockage elements 224 also are rolled in the lumen closed position as shown in FIG. 40. FIG. 40 also illustrates how the body 104 collapses to accommodate the natural collapse of a bodily lumen 5. As the pressure increases in the lumen 110, the device expands as shown in FIG. 41. Further increases in pressure will cause the blockage devices 224 to unroll and extend along the sidewall 105 thereby un obstructing the lumen 110. FIG. 41 also illustrates how the blocking devices may be oriented in different configurations to further modify the pressure and flow response of the device.

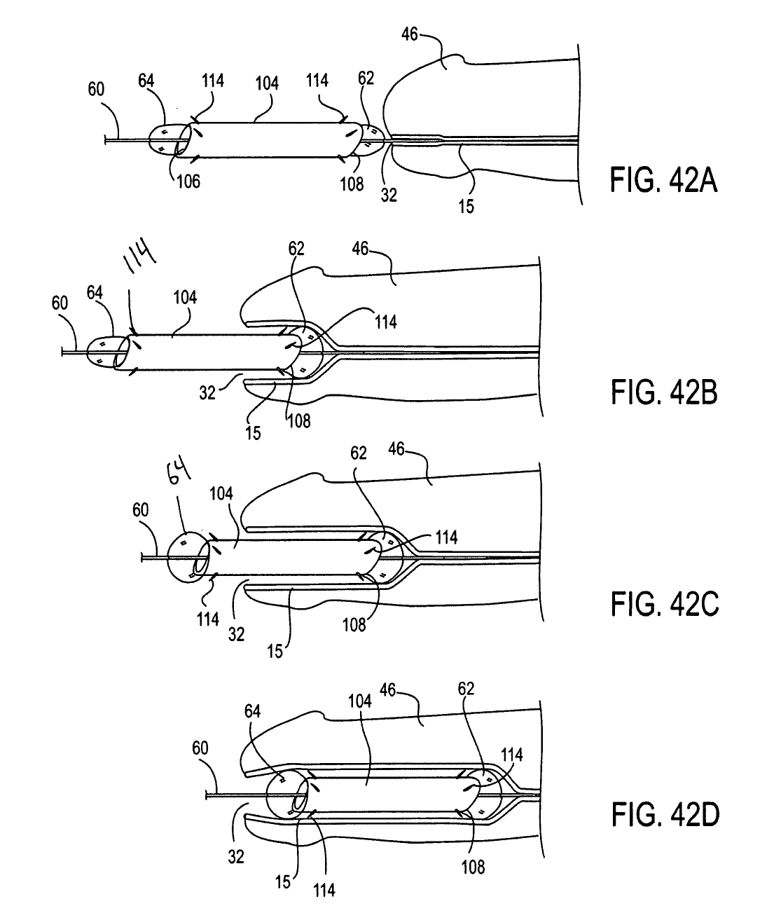

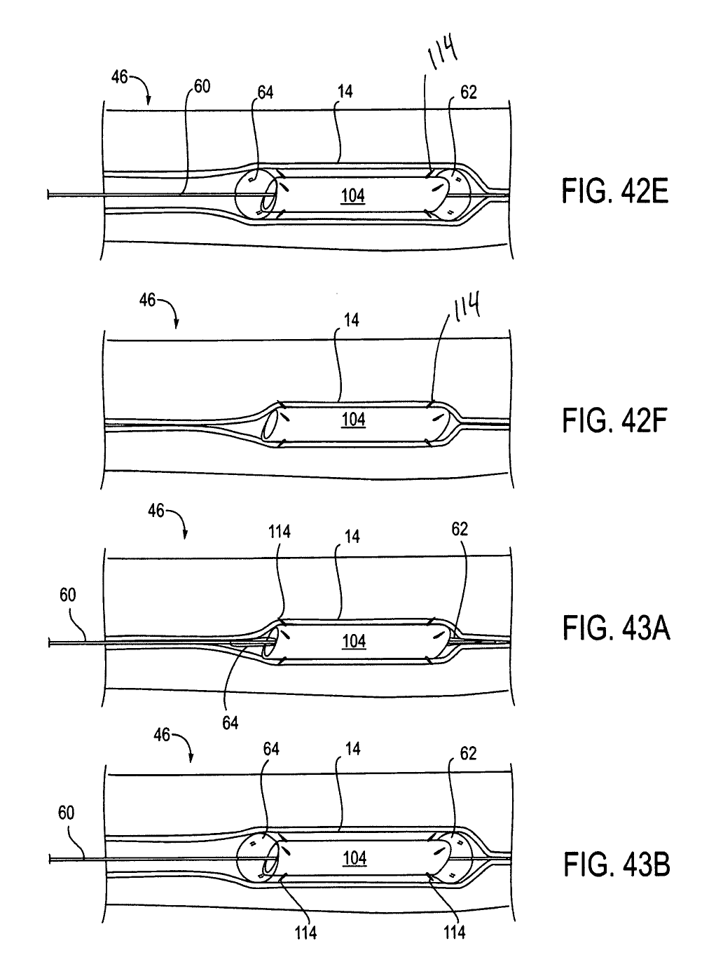

FIGS. 42A-43F illustrate the delivery of an embodiment of the device 100 using a catheter 60. The device 100 includes fixation elements 114 to anchor the device within the body lumen 5. In this illustrative example, the body lumen 5 will be the urethra 15 of a human male. As a preliminary manner, a local anesthetic may be applied into the urethra 15 via the external orifice 32 alone or in combination with coating the device with a lidocaine gel. Lubricants will also likely be used to aid in delivery and removal. Hormone cream can be used in conjunction with this device as well as the drug eluting concepts described below to prevent or delay mucosal ulceration. Turning now to FIG. 42A, the device 100 is delivered and removed by means of a flexible catheter 60 that, in the illustrative embodiment, includes two balloons 62, 64. The catheter 60 is inserted through the device lumen 110. In the illustrated embodiment, two balloons are provided. Balloon 64 at the proximal end and balloon 62 at the distal end of the device body. The end of the catheter 60 is inserted into the body lumen 15 and the distal balloon 62 is inflated to dilate the body lumen 5 beyond the penetrating members of the anchoring system. As shown in FIG. 42 A, the distal balloon 62 is inserted into the external orifice 32 to dilate the urethra 15. The balloon 62 may be partially inflated as shown or may be inserted into the urethra prior to inflation. Next, as shown in FIG. 42B, the distal balloon 62 is inflated to dilate the urethra so that the fixation elements 114 to not penetrate or damage the walls. Next, as shown in FIG. 42C, the device body is further advanced into the urethra. Next, as shown in FIG. 42D, the proximal balloon 64 is used to maintain the urethra in a dilated condition in the vicinity of the fixation elements 114 on the proximal end of the device. With the balloons 62, 64 dilating the urethra 15, the device is advanced to the implantation site 90 as shown in FIG. 42E. The position of the device 100 may be confirmed using any suitable medical device imaging modality such as X-ray, CT scan, endoscopic visualization, ultrasound, MRI, palpitation and the like. To set the fixation elements, the balloons 62, 64 are now deflated and the urethra is allowed to coapt or collapse onto the device 100. As the urethra coapts, the fixation elements penetrate the walls and anchor the device as shown in FIG. 42F. The anchoring members 114 are illustrated as angled in opposition so that the device 100 is prevented from migrating in either direction of the urethra 15. Once delivered, the device 100 is anchored through some temporary or permanent means as described in greater detail below. Anchoring or fixation may be accomplished by any suitable means to secure a device into a bodily lumen 5, including spikes that point in, out, or can be twisted to be seated into the bodily lumen 5. Depending upon the design of the fixation element, the fixation elements or element may remain within the wall of the urethra or may pierce the urethra wall. With the balloons 62, 64 deflated, the catheter 60 is withdrawn and the device is implanted in the urethra 15 as shown in FIG. 42F.

In one embodiment, the device 100 contains a built-in balloon or balloons similar to balloons 62, 64. Additionally or in the alternative, a balloon may be provided in the middle of the device 100 to aid in preventing the distal device from other extending to provide clearance for the proximal fixation elements (see FIG. 42C). Moreover, the distal balloon 62 could have a different shape such a plurality of elongate longitudinal elements extending along the outside of the device 100. These finger-like balloons would distend the bodily lumen 5 clear of all fixation elements. Additionally or alternatively, the finger balloon may be formed on and be a part of the device 100 instead of the catheter 60.

Other balloon configurations are possible. In one alternative embodiment, only a distal balloon is used so that anchors 114 remain clear the urethra and the device may be advanced. In another alternative, distal and proximal balloon that expand along the longitudinal axis of the device body are used so that anchors clear the urethra and the device may be advanced. In another alternative, the distal and proximal balloons expand along the longitudinal axis of the device body to enclose the fixation elements so that anchors clear the urethra and the device may be advanced. In yet another alternative, distal, proximal and device body mounted balloons so that anchors clear the urethra and the device may be advanced. Expansion of the balloons is merely exemplary. It is to be appreciated that a coil, cage or other expandable structure may be used in place o the balloons to expand the lumen.

Removal of the device 100 will now be described. As shown in FIG. 43A, the catheter 60 is inserted through the urethra to the implantation site 90 with the balloons 62, 64 deflated. Once in position adjacent to the device 100, the balloons 62, 64 are inflated as shown in FIG. 43B to dilate the urethra beyond the penetrating anchoring members 114. Thereafter, the catheter 60 with the device 100 attached is withdrawn as shown in FIGS. 42D, 42C, 42B and 42A.

As described above in the insertion and recovery process, anchoring the device 100 into a bodily lumen occurs when the implant balloons are deflated or after a twist motion depending upon fixation element design. Alternatively, fixation may occur as described above with regard to FIGS. 2-4. In some embodiments, the anchoring mechanism includes a plurality of angled penetrating members 114 that fix the device body 104 in position within the bodily lumen 5.

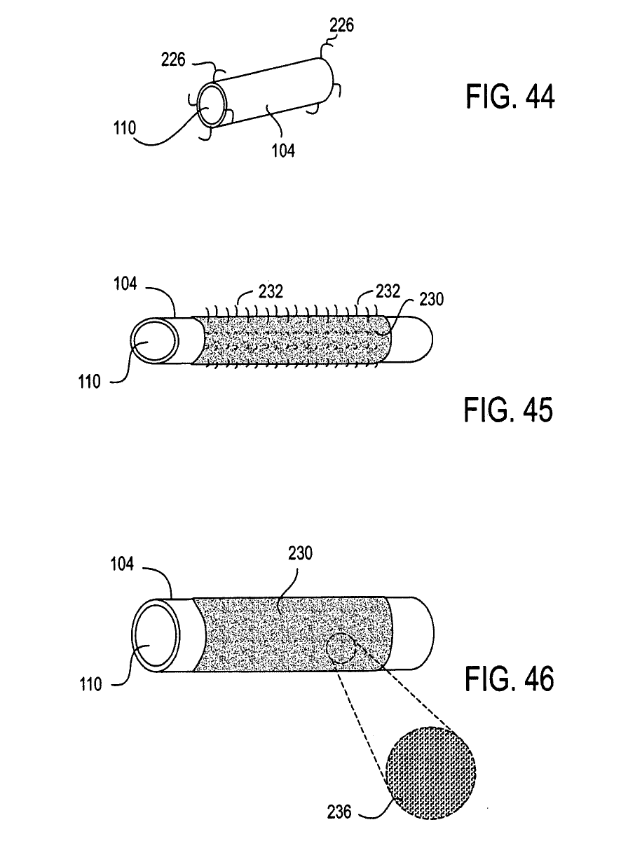

Numerous variations and combinations of fixation elements are possible. For example, spikes, tines, barbs and the like may be angled perpendicularly, angled inward, or angled outward in reference to the body 104. Additionally, non-penetrating fixation techniques based, for example, on suction cups, the use of vacuum, and other features on the device body 104 may also be used. The number and position of the fixation elements 114 relative to one another and the body may vary but are used to prevent migration of the device 100 within the urethra (upstream or downstream) or into the bladder, for example. As shown in FIG. 44. The penetrating members 226 can also be oriented relative to the urethral endothelial surface so that when rotational movement is applied to the device body 104, the anchoring members 226 will then penetrate the urethra 15. Additionally, as described above with regard to FIGS. 3A-3D, the fixation members 114 may be disposed within the sidewall 110 while being moved into position within the body lumen 5. Then, once in position, the fixation devices 114 are deployed to engage with the body lumen 5 to anchor the device 100 in place. Any conventional fixation element deployment technique may be used, such as, for example, spring loaded, pneumatically actuated, temperature activated (such as when shape memory alloy fixation elements 114 are used) or other techniques suited to the type of fixation element used. Additionally, bio-adhesives may also be used to secure the device 100 within the lumen 5.

In addition to the large scale fixation elements described so far, fixation may also occur on a smaller scale. Consider FIG. 45 that illustrates a backing or structure 230 on the device body 104. The structure 230 has an array of micro-barbs or pins 232 distributed across its surface. When inserted into the bodily lumen 5, the pins 232 engage with the lumen 5 to anchor the device 100 in place. FIG. 46 illustrates a similar micro or small scale fixation device. In this illustrative embodiment, the backing 230 has a a plurality of micro-hooks 236 similar to the hook fasteners used in Velcro.

Additionally, magnets may be used to anchor the device in a body lumen. A magnet 240 encased in biocompatible material such as prolene or other material to induce scarring of around the magnet 240 to secure it into position. The magnet 240 is inserted into position adjacent to an implantation site in a body lumen 5. A magnet pair is then created between the magnet 240 and another magnet or ferrous component on the device 100 when the device 100 is in the correct position in the lumen 5. FIG. 47 illustrates one embodiment where the magnet 240 has been implanted into a suitable implant site near the female urethra 15. Here, a transvaginal needle insertion technique was used to implant the anchor magnet with a soft tissue anchor. The device 100 on catheter 60 has been moved into position and is coupled to the magnet 240 to fix the device 100 in position within the urethra 15. In an alternative embodiment, the magnet 240 partially or completely encircles the urethra 15. A similar technique may be used to implant the magnet 240 in a male using a transperineal approach.

FIG. 48 illustrates another alternative anchoring device where a spiral or coil element 245 is used to secure the device 100 within the bodily lumen 5. Complete spirals need not be used. Partial or only a single revolution spiral may also be used. The coil 245 may be on one end, or both ends as illustrated in FIG. 48. The coil 245 may alternatively have a partial coil or hoop or a complete coil as illustrated in FIG. 48. Additionally, the coil 245 may optionally include one turn, only a partial turn or multiple turns as illustrated in FIG. 48.

FIG. 49 illustrates a balloon 250 along the device body 104. When inflated, the balloon 250 secured the device into the bodily lumen 5. A balloon or other expandable structure around the body 104 expands to hold the device 100 in place. In this embodiment, the balloon 250 also seals the lumen of the urethra so that flow is through the device lumen 110. Alternatively or in addition, balloons may be provided on one or both ends of the device body 104 and expanded to hold the device 100 in place.

Further to the concepts described above in FIGS. 3A-3D, the anchoring elements may be contained within the device (such as in sidewall 105 or lumen 110, for example) and then externally activated to engage with surrounding tissue. In one example, remote activation of a lever or mechanism on the catheter or outside the body lumen 5 is used to advance the fixation element or elements to engage. Remote activation of the mechanism in reverse is used to during the withdrawal sequence. In addition, shape memory alloys (SMA) fixation elements with a memory position outside of the device that is disengaged and a shape memory position that is engaged when within the device is in the body lumen at body temperatures. To remove, a appropriately temperature controlled balloon may be inserted into the device body to adjust the temperature of the fixation elements to reverse them into a disengaged position. Once disengaged and withdrawn into the device body, the device body is removed.

In other embodiments, the device 100 or components of the device may be coated with drugs, or agents to produce a pharmacological effect in the body lumen 5. Drug eluting examples include coatings comprising: estrogen, anti-inflammation agents, and/or antibiotic agents. Drug coated drug eluting embodiments of the device 100 may be appreciated through reference to U.S. Patent Application Publication 2006/0264697 titled “Drug Elution for Implantable Incontinence Devices” to Timm, et al, the entirety of which is incorporated herein by reference in its entirety.

While certain embodiments of the present invention have been described in the illustrative example of controlling urine flow from the bladder, it is to be appreciated that the invention is not so limited. Embodiments of the invention may find applicability in not only urinary incontinence but may be applied to fecal incontinence or GERD or to other structures with a lumen in the human or mammal body where flow is to be regulated. Embodiments described herein may be adapted and configured for insertion into and regulation of flow within the colon, the esophagus and other portions of the alimentary canal. Additional other details regarding construction, materials, operation and alternatives are in U.S. Pat. No. 6,988,983 titled “Implantable Self-Inflating Attenuation Device,” U.S. Patent Application Publication 2006/0047180 titled “Artificial Sphincter,” U.S. Patent Application Publication 2006/0205997 titled “Urinary Flow Control Device and Method,” U.S. Pat. No. 6,540,665 titled “Urinary Continence Device,” U.S. Pat. No. 6,027,442 titled “Urethra Control Device” each of which is incorporated herein by reference in its entirety.

What is claimed is:

1. A device completely disposable within a bodily lumen of a patient and for controlling flow through the bodily lumen, comprising:
a device body having a proximal end, a distal end and a device lumen within the device body extending from the proximal end to the distal end, the device body completely disposed within the bodily lumen so that all flow through the bodily lumen flows through the device lumen; and
a fixation element extending from the outer wall of device body adapted and configured to engage with the wall of the bodily lumen; wherein,
the device body is adapted and configured to move from a position where the device lumen blocks flow through the bodily lumen to a position where the device lumen allows flow through the bodily lumen.

2. The device of claim 1 wherein the device body moves in response to increased intraluminal pressure applied anatomically proximal to the device.

3. The device of claim 2 wherein the increased pressure is applied for longer than three seconds.

4. The device of claim 1 wherein at least one of the proximal device end or the distal device end is angled to facilitate movement of the device between positions.

5. The device of claim 1 wherein the fixation element withdraws into the sidewall of the device body or the device lumen.

6. The device of claim 1 wherein the fixation element is a balloon.

7. The device of claim 1 wherein the fixation element has a stowed condition where the fixation element is engaged with the bodily lumen wall and a deployed condition where the fixation element engages with the bodily lumen wall.

8. The device of claim 1 wherein the bodily lumen is the urethra.

9. The device of claim 1 wherein the device lumen is non-linear.

10. A device completely disposable within a bodily lumen of a patient and for controlling flow through the bodily lumen, comprising:
a device body having a proximal end, a distal end and a device lumen within the device body extending from the proximal end to the distal end, the device body completely disposed within the bodily lumen so that all flow through the bodily lumen flows through the device lumen; and
one or more valves within the device lumen adapted and configured to move between a position to block flow through the bodily lumen and a position to allow flow through the bodily lumen.

11. The device of claim 10 further comprising a fixation element extending from the outer wall of device body adapted and configured to engage with the wall of the bodily lumen.

12. The device of claim 10 wherein the one or more valves within the device lumen includes a magnetically activated rotary valve.

13. The device of claim 10 wherein the one or more valves within the device lumen includes a flap valve.

14. The device of claim 10 wherein the one or more valves within the device lumen are angled relative to the device lumen.

15. The device of claim 10 wherein the one or more valves within the device lumen move between a position to block flow through the bodily lumen and a position to allow flow through the bodily lumen by moving a fluid from the interior of a valve to a cavity in the sidewall of the device body.

16. The device of claim 10 wherein the one or more valves within the device lumen is a valve have a pair of disks mounted on, in or within the sidewall of the bodily lumen, the pair of disks rotatable relative to one another to move between positions.

17. A method for implanting a device within a body lumen, comprising: placing a device having a device body and fixation elements extending from the device body on a catheter;
dilating the body lumen such that the device body and the fixation elements may advance through the dilated body lumen without the fixation elements engaging the wall of the body lumen;
advancing the device to an implant site within the body lumen; and engaging the walls of the body lumen with the fixation elements.

18. The method of claim 17 wherein the dilating step is performed by inflating a balloon or expanding a structure on the catheter.

19. The method of claim 18 wherein the balloon is distal to the distal end of the device.

20. The method of claim 17 wherein the dilating step is performed by inflating a balloon or expanding a structure on the device.

21. The method of claim 17 wherein the engaging step is performed by rotating the device relative to the bodily lumen.
